(19) United States
(12) Patent Application Publication
(10) Pub. No.: US 2008/0177071 A1
(43) Pub. Date: Jul. 24, 2008

(54) Process for the preparation of 2- (2- (4- (bis (4-flourophenyl) methyl) -pipe razin-1-yl) ethoxy acetic acid derivatives or corresponding salt forms thereof and intermediates therefor

(76) Inventors: Ousmane Diouf, Flines Lez Raches (FR); John Surtees, Jezus-Eik (Overijse) (BE)

Correspondence Address: WENDEROTH, LIND & PONACK, L.L.P., 2033 K STREET N. W., SUITE 800, WASHINGTON, DC, 20006-1021, US

(21) Appl. No.: 11/976,839

(22) Filed: Oct. 29, 2007

Related U.S. Application Data
(62) Division of application No. 10/484,838, filed on Jan. 26, 2004, now abandoned.

(30) Foreign Application Priority Data
Jul. 26, 2001 (EP) EP01118131.0

(51) Int. Cl. C07D 295/088

(52) U.S. Cl. 544/399

(57) ABSTRACT

The present invention relates to a process for the manufacture of 2-{2-[4-(bis(4-fluorophenyl)methyl)-1-piperazinyl]ethoxy}acetic acids, amides or related derivatives, of the general formula (I) wherein: Y represents hydroxy or —NR₁R₂; R₁ and R₂ each independently represent hydrogen or C?1-4#191 alkyl; m is 1 or 2, and n is 1 or 2, as well as the non-toxic, pharmaceutically acceptable salts and mixtures thereof. The present invention concerns also a polymorphic form of efletirizine.

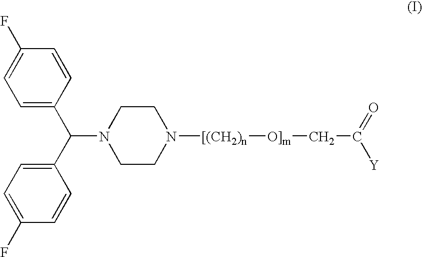

The present invention relates in a first aspect to a new and improved process for the preparation of 2-(2-[4-(bis(4-fluorophenyl)methyl)-1-piperazinyl]ethoxy)acetic acid derivatives or corresponding salt forms thereof. Said compounds, and in particular 2-(2-(4-[bis(4-fluorophenyl)methyl)-1-piperazinyl]ethoxy)acetic acid commonly known as efletirizine, have been proven useful as therapeutic agents for the treatment of allergic diseases and other disorders.

In a second aspect, the present invention relates to a new polymorphic form of efletirizine. A process for the preparation of this new polymorphic form and pharmaceutical compositions containing it are also comprised within this invention.

Efletirizine has been found to possess excellent antihistaminic properties. It belongs to the pharmacological class of second generation histamine H₁-receptor antagonists and shows in vitro high affinity and selectivity for H₁-receptors. Efletirizine is useful as an antiallergic, antihistaminic, bronchodilator and antispasmodic agent. Recent clinical studies have shown the utility of efletirizine when administered in the form of a nasal spray for the treatment of allergic rhinitis and rhino-conjunctivitis (J.-F. Dessanges et al., Allergy and Clin. Immunol. News (1994), Suppl. no. 2, abstract 1864; C. De Vos et al., Allergy and Clin. Immunol. News (1994). Suppl. no. 2, abstract 428). Another recent clinical pharmacological study has shown that efletirizine gives unexpectedly good results in the treatment of urticaria, atopic dermatitis and pruritis.

In the light of the versatility of efletirizine as a powerful drug for the treatment of allergic and other diseases, a new, low cost, easy to perform and high yielding process for its preparation is desirable.

2-{2-[4-[bis(4-fluorophenyl)methyl)-1-piperazinyl]ethoxy}acetic acid or efletirizine, in the form of its dihydrochloride salt has the following formula:

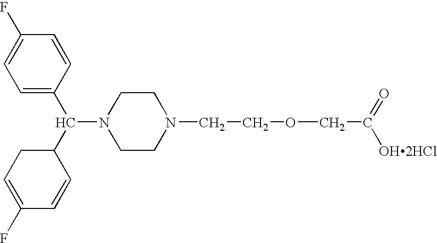

Efletirizine is encompassed within the general formula of European Patent No. 0 058 146 and may be prepared according to the general process described in this patent. Said process for the synthesis of 2-(2-[4-(diphenylmethyl)-1-piperazinyl]ethoxy)acetic acid derivatives comprises reacting a 1-(diphenylmethyl)piperazine derivative with methyl(2-chloroethoxy)acetate or 2-(2-chloroethoxy)acetamide to form a methyl 2-{2-[4-(diphenylmethyl)-1-piperazinyl]ethoxy}-acetate or a 2-{2-[4-(diphenylmethyl)-1-piperazinyl]ethoxy}acetamide, respectively. Thus the formed methyl ester or acetamide is then subjected to basic hydrolysis followed by acidification and isolation of the free carboxylic acid. This material is then transformed into its dihydrochloride salt.

European Patent No 1 034 171 describes two pseudo-polymorphic forms of efletirizine. There is a desire for an alternative economical and high yielding process for the synthesis of efletirizine.

According to the present invention, a new process for the synthesis of 2-{2-[4-(bis(4-fluorophenyl)methyl)-1-piperazinyl]ethoxy}acetic acids and their corresponding salt forms is provided. In particular, said new process can be employed for the synthesis of efletirizine and markedly overcomes several disadvantages of the known methods. In a first aspect, the present invention concerns a process for the manufacture of 2-{2-[4-(bis(4-fluorophenyl)methyl)-1-piperazinyl]ethoxy}acetic acids, amides and related derivatives of the general formula (I)

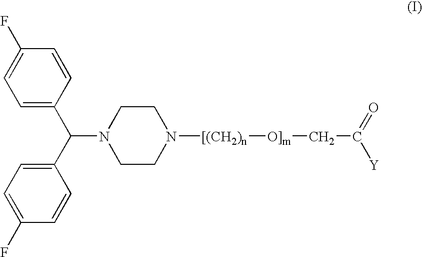

wherein: Y represents hydroxy or —NR¹R²; R¹ and R² each independently represent hydrogen or C₁₋₄ alkyl; m is 1 or 2, and n is 1 or 2,
as well as non-toxic, pharmaceutically acceptable salts and mixtures thereof, characterized by
(a) reacting compound of formula (II)

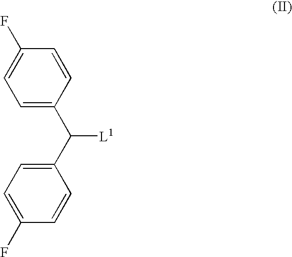

wherein L¹ represents a leaving group, with a compound of formula (III)

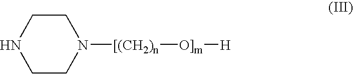

wherein n and m are defined as above, in the presence of a base and an inert solvent, and (b) reacting the corresponding compound of formula (IV) thus obtained

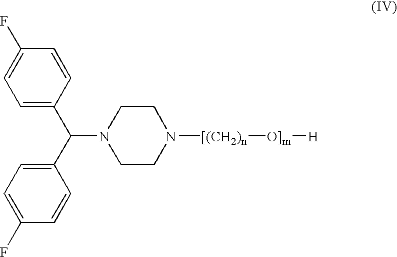

with a compound of formula (V)

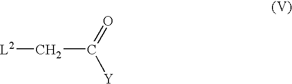

wherein L² represents a leaving group and Y is defined as above, in the presence of an inert solvent and a proton acceptor.

The term “leaving group”, as used herein, has the same meaning by the skilled man (Advanced Organic chemistry: reactions, mechanisms and structure—Third Edition by Jerry March, John Wiley & Sons Ed.: 1985 page 179) and represents a group which is part of and attached to a substrate molecule. In a reaction where the substrate molecule undergoes a displacement reaction (with for example a nucleophile), the leaving group is then displaced. Examples of leaving group are alkoxy, alkylthio, trimethylamino, methylsulfinyl, methylsulfonyl or halogen. Preferably the leaving group is halogen or a sulfonic ester group. The term “halogen”, as used herein, includes an atom of Cl, Br, F, I. The term “sulfonic ester group”, as used herein, has the same meaning by the skilled man (Advanced Organic chemistry: reactions, mechanisms and structure—Third Edition by Jerry March, John Wiley & Sons Ed.; 1985 pages 311-312) and represents a reactive ester. Since hydroxide does not leave readily from ordinary alcohols, it must be converted to a group that does leave, one way is conversion to a reactive ester, such as a sulfonic group. The sulfonic acids groups tosylate (paratoluenesulfonates) and mesylate (methanesulfonates) can be used. The term “sulfonic acid”, as used herein, represents a group of the formula —SO₃H.

According to a preferred embodiment, the present invention is particularly suited for the manufacture of a compound of formula (I) as described above, wherein n is 2. According to a preferred embodiment, the present invention is particularly suited for the manufacture of a compound of formula (I) as described above, wherein m is 1.

Particularly preferred is 2-{2-[4-[bis(4-fluorophenyl)methyl)-1-piperazinyl]ethoxy}acetic acid (also known as efletirizine). These are especially preferred as dihydrochlorides.

According to another preferred embodiment, in the compound of formula of (II), L¹ represents chlorine.

According to another preferred embodiment, in the compound of formula of (V), L² represents bromine.

Suitable bases for use in the step a) are alkali metal carbonates, hydroxides and organic tertiary amines. Sodium and potassium carbonate are preferred.

As proton acceptor for use in the step b) alkali metal hydrides, alkali metal hydroxides, alkali metal alkoxides and alkali metals are preferred. Sodium hydride and sodium methoxide are especially preferred.

As solvent, any chemically inert solvent such as aliphatic and aromatic hydrocarbons, ethers, amides and alcohols of low reactivity may be used. Preferred solvents are hexane, toluene, methyl ethyl ketone (MEK), dimethoxyethane (DME), tetrahydrofurane (THF), dimethylformamide (DMF) or tert-butanol.

The process of this invention is particularly useful for the production of 2-{2-[4-[bis(4-fluorophenyl)methyl)-1-piperazinyl]ethoxy}acetic acid (efletirizine) in the form of its dihydrochloride.

In another embodiment, the invention concerns a process for the manufacture of a compound of formula (IV)

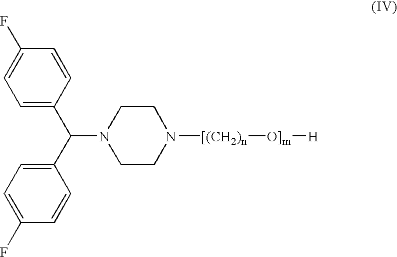

wherein m is 1 or 2, and n is 1 or 2, as well as non-toxic, pharmaceutically acceptable salts and mixtures thereof, characterized by reacting a compound of formula (II)

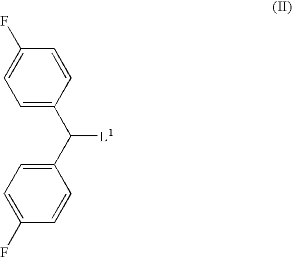

wherein L¹ represents a leaving group, with a compound of formula (III)

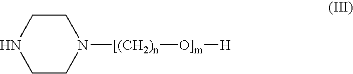

wherein n and m are defined as above, in the presence of a base and an inert solvent.

In another embodiment, the invention concerns also a process for the manufacture of 2-{2-[4-(bis(4-fluorophenyl)methyl)-1-piperazinyl]ethoxy}acetic acids, amides and related derivatives of the general formula (I)

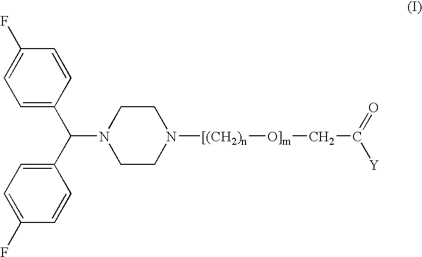

wherein: Y represents hydroxy or —NR¹R²; R¹ and R² each independently represent hydrogen or C₁₋₄ alkyl; m is 1 or 2, and n is 1 or 2, as well as non-toxic, pharmaceutically acceptable salts and mixtures thereof, characterized by reacting a compound of formula (IV)

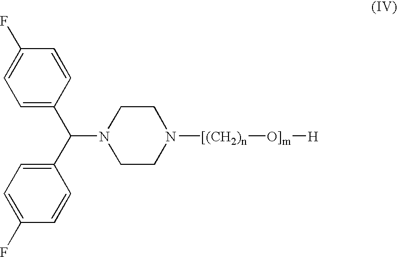

wherein m and n are defined as above,
with a compound of formula (V)

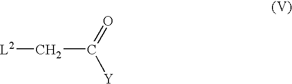

wherein L² represents a leaving group and Y is defined as above, in the presence of an inert solvent and a proton acceptor.

The following description relates to the manufacturing process of efletirizine. However, it will be apparent to those skilled in the art that said compound can be interchanged with any other compound encompassed by the general formula (I) as given above.

In general, the new manufacturing process for efletirizine consists of two major steps. The first step is the reaction of bis(4-fluorophenyl)methylchloride with N-(2-hydroxyethyl)piperazine. The substitution of the chlorine atom of the bis(4-fluorophenyl)methyl moiety is performed in the presence of a base because hydrogen chloride (HCl) is generated during the reaction. This HCl would tend to interact with the free amine functionality of the piperazine starting material, rendering it inactive and therefore has to be neutralised in order to achieve optimum conversion (and therefore yield).

Both mineral and organic bases can be used for said purpose, such as alkali metal carbonates, hydroxides and organic tertiary amines. For convenience of work up (product isolation) and lower cost, it is generally fairly preferable to carry out the reaction in the presence of alkali metal carbonates (such as potassium and sodium carbonates). The most appropriate organic base is triethylamine.

An alternative is to use the starting materials or the final products themselves as base, since they contain basic nitrogen (i.e. amine) functionality in the form of the piperazine moiety—as noted above, these would react with excess HCl. In the case where the starting material acts as base, at least two equivalents of it are necessary to bring the reaction to completion. Although this is in principle an alternative approach, it is preferred to avoid this methodology since it leads to waste of more expensive starting material and/or to necessity of recycling.

The second step in the manufacturing of efletirizine consists of several stages. The second step is classically identified as a “one-pot reaction” since all stages may be realised successively and/or simultaneously in the same reactor.

First a proton acceptor is used to deprotonate the starting material, 2-{2-[4-[bis(4-fluorophenyl)methyl)-1-piperazinly]ethanol. In principle, any proton acceptor known to those skilled in the art can be used, such as alkali metal hydrides, alkali metal hydroxides, alkali metal alkoxides and alkali metals. For the preparation of efletirizine, both sodium hydride (NaH) and sodium methoxide (CH₃ONa) are preferred.

Use of CH₃ONa leads to the formation of methanol, which is easily removed by distillation or evaporation under reduced pressure. From the industrial safety viewpoint CH₃ONa is preferable to NaH. These deprotonation reactions are depicted in scheme 1 below.

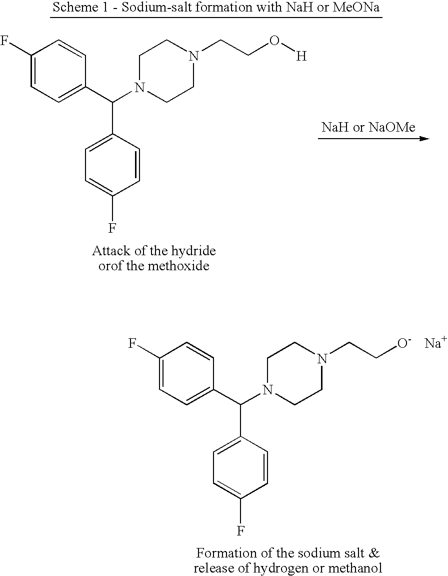

In a following step, the sodium alkoxide obtained is reacted with a salt of a haloacetic acid, such as sodium bromoacetate or its chloro analogue, and not directly with the corresponding haloacetic acids. It is obvious that the alkoxide would simply be inactivated in the presence of an acid by a classical acid-base reaction resulting in its re-protonation to the corresponding alcohol.

To circumvent this inactivation the halogenoacetic acid derivative may be used in a corresponding salt form. This is why in the case of bromoacetic acid, it is preferred first to treat the acidic derivative with sodium hydride before reacting it with the alkoxide obtained during step 1. In the case of chloroacetic acid, the sodium chloroacetate derivative is already commercially available. The condensation reaction between the alkoxide and sodium bromoacetate is shown in detail in scheme 2 below.

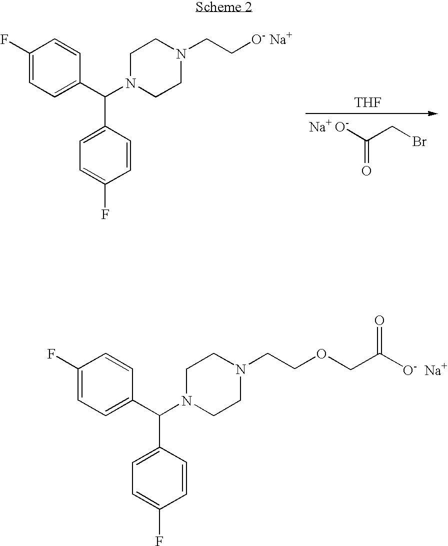

The obtained efletirizine can easily be converted into its dihydrochloride form. Therefore, the sodium salt of efletirizine obtained as end product in the second step of the manufacturing process is dissolved in water and the resulting solution then acidified, preferably with aqueous hydrochloric acid solution (preferably to a pH of about 1).

The new manufacturing process of this invention has several advantages compared to the prior art method as described above in the background of this invention.

For instance, the process of this invention no longer utilises piperazine as such but instead a piperazine intermediate is used. Said piperazine intermediate e.g. N-(2-hydroxyethyl)piperazine or a similar compound of formula (III), comprises only one single reactive nitrogen compared to piperazine itself having two reactive sites. In the prior art method said piperazine is used both as reaction product and as base at the same time. The excess of piperazine has to be recovered at the end of the reaction requiring an expensive recycling process. On the contrary, in the process of this invention, inexpensive mineral bases such as sodium carbonate can be used, with no necessity for recycling. Furthermore, condensation of bis(4-fluorophenyl)methylchloride (DFBCl) with N-(2-hydroxyethyl)piperazine requires very little excess of said piperazine intermediate (0.5 equivalents in excess), whereas in the prior art method quite a large excess of piperazine is needed (4 equivalents in excess).

As a result, the manufacturing process of this invention is much cheaper and more economical compared to the prior art method as described in EP Patent No. 58 146 and other methods currently employed. In addition, in this invention 2-chloroethoxy acetamide (CEA) is not used, again lowering the production cost significantly. Other starting materials for use in the process of this invention are also inexpensive.

The new process is less time consuming, uses less expensive starting materials and does not require any recycling process to recover unused reagents. Therefore, this process is economically more favorable compared to the manufacturing process currently employed. Furthermore, very high yields can be obtained by this process, constituting a considerable technical advantage with respect to other known methods in particular in the process described in European Patent No. 0 058 146.

In another aspect, the present invention provides a process for the preparation of a new polymorphic form of efletirizine. Said polymorphic form is characterized by its particular X-ray diffraction pattern as described in full below.

The invention also encompasses said new polymorphic form itself, particularly as obtainable by the process according to the invention, as well as pharmaceutical compositions comprising said form in association with a pharmaceutically acceptable adjuvant, diluent or carrier.

A preferred method for obtaining said new polymorphic form of efletirizine comprises following steps:

(a) precipitating of the compound obtained by the process according to the invention in its dihydrochloride form.
(b) washing the obtained precipitate with a suitable organic solvent.
(c) re-dissolving the washed precipitate in an aqueous medium.
(d) adjusting the pH of the aqueous medium to about 7 with a suitable base or buffer.
(e) extracting the re-dissolved product with a suitable organic solvent,
(f) washing the obtained organic layer,
(g) drying said obtained organic layer, preferably over magnesium sulphate, followed by acidification with HCl, and
(h) finally precipitating and drying the obtained dihydrochloride salt.

The organic solvent used in steps b) and e) is preferably a ketone or an ether. Most preferably methyl ethyl ketone (MEK) is used

The base or buffer used in step d) is generally an inorganic base, preferably an alkali metal carbonate or hydroxide. Most preferably potassium carbonate is used.

The washed precipitate obtained after step b) is preferably dried before being re-dissolved in an aqueous medium according to step c).

Said new polymorphic form of efletirizine can be characterized by its crystallographic X-ray diffraction pattern and presents peaks at 2θ values (±0.5) of: 7.000°; 8.095°; 12.000°; 13.645°; 14.085°; 14.315°; 14.870°; 16.460°; 17.295°; 18.255°; 18.755°; 19.470°; 20.575°; 20.890°; 21.660°; 22.210°; 22.890°; 23.390°; 24.210°; 24.580°; 25.130°; 26.775°; 27.855°; 28.815°; 29.820°; 30.255°; 31.460°; 32.145°; 32.890°; 33.830°; 34.695°; 35.940°; 38.135°; 39.670°; 43.065°; 44.335°; 46.210; 48.720.

In another embodiment, the invention concerns also a compound obtained by the process described above, such as intermediates.

According to a preferred embodiment, such a compound is a compound of formula (IV)

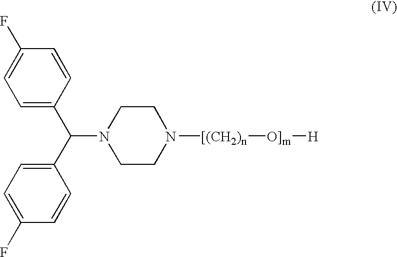

wherein m is 1 or 2, and n is 1 or 2, as well as non-toxic, pharmaceutically acceptable salts and mixtures thereof.

The present invention will be better understood from the following examples which only serve to illustrate the invention and therefore should not be taken to limit the scope thereof.

### EXAMPLES

### Example 1

### Preparation of 2-(2-[4-[bis(4-fluorophenyl)methyl)-1-piperazinyl]ethanol

In a 300 ml reactor equipped with a reflux condenser, introduce 0.016 mole, 2.65 g of potassium iodide (15% compared to bis(4-fluorophenyl)methylchloride (DFBCl)), 0.158 mole of sodium carbonate (Na₂CO₃) (1.5 equivalents compared to, DFBCl), 100 ml of methyl ethyl ketone (MEK) (4 volumes compared to DFBCl and 0.158 mole, 20.6 g of N-(2-hydroxyethyl)piperazine (1.5 equivalents). Heat up the reaction mixture to 90° C. Once reflux has started, add dropwise 0.105 mole, 25 g of DFBCl over a 30 minutes period and let the mixture stir for 2 hours. Evaporate the MEK, resuspend the residue in 400 ml of water and extract twice with 100 ml of diisopropylether (DIPE). Add 100 ml of water to the organic phase and adjust the pH of the aqueous phase to 2.4 with aqueous HCl 37%. Wash the aqueous phase 3 times with 100 ml of toluene, then basify the aqueous phase with sodium hydroxide 50%. Extract the aqueous phase 3 times with 100 ml of toluene. Wash the toluene phase with 100 ml of demineralized water and eliminate residual water by azeotropic distillation. Evaporate the toluene.

Following this process, 29 g of 2-[2-[4-[bis(4-fluorophenyl)methyl)-1-piperazinyl]ethanol was obtained, i.e. a yield of 83% (HPLC analysis 99.9%).

### Example 2

### 2-[2-[4-[bis(4-fluorophenyl)methyl]-1-Piperazinyl]ethoxy]acetic acid

### Example 2A

### Reaction in the Presence of NaH as Proton Acceptor

5 g of the obtained 2-(2-[4-[bis(4-fluorophenyl)methyl)-1-piperazinyl]ethanol in example 1 is introduced into a reactor in the presence of anhydrous THF (50 ml) and NaH (2.5 equivalents). This is heated at 50° C. and bromoacetic acid (1.3 equivalents) is added. The reaction is allowed to proceed overnight. Evaporate the THF, resuspend the residue in 100 ml of water (formation of 3 phases). Recover the intermediate phase, and take it up in 100 ml of water. Acidify with a solution of HCl 37% until homogenization of the medium (pH 1). Wash the aqueous phase with three times 100 ml toluene, concentrate the water until appearance of a white precipitate. Let it precipitate completely overnight at 4° C. in the refrigerator. Filter the precipitate, wash it with MEK and dry it.

Following this process, 2-[2-[4-[bis(4-fluorophenyl)methyl]-1-piperazinyl]ethoxy]acetic acid dihydrochloride was obtained with a yield of 96% and purity of 97.4%.

### Example 2B

### Reaction in the Presence of CH₃ONa as Proton Acceptor

In a 300 ml reactor, introduce 0.09 mole. 30 g of 2-(2-[4-[bis(4-fluorophenyl)methyl)-1-piperazinyl]ethanol such as described in example 1, 120 ml of toluene and carefully add 12.15 of sodium methoxide (2.5 equivalents) slowly. Allow to stir for 10 hours at room temperature then evaporate the solvent. Take up the residue in 160 ml of tetrahydrofuran (THF) and add 16.4 g of sodium bromoacetate (1.3 equivalents). Heat up the mixture to 50° C. and let it stir overnight. Evaporate the THF, resuspend the residue in 100 ml of water (formation of 3 phases). Recover the intermediate phase, and take it up in 100 ml of water. Acidify with a solution of HCl 37% until homogenization of the medium (pH 1). Wash the aqueous phase with three times 100 ml toluene, concentrate the water until appearance of a white precipitate. Let it precipitate completely overnight at 4° C. in the refrigerator. Filter the precipitate, wash it with MEK and dry it.

Following this process, 37 g of 2-(2-[4-[bis(4-fluorophenyl)methyl)-1-piperazinyl]ethoxy]acetic dihydrochloride was obtained, i.e. a yield of 87.7% (HPLC analysis 97.4%).

### Example 3

### Preparation of a new polymorphic form of 2-[2-[4-[bis(4-fluorophenyl)methyl]-1-piperazinyl]ethoxy]acetic acid dihydrochloride (efletirizine)

The dried powder obtained from Example 2B was re-dissolved in water, the pH of the solution was brought to 7 using an aqueous solution of potassium carbonate and extracted with methyl ethyl ketone. The organic layer was washed with water, dried over magnesium sulphate, filtered and gaseous HCl was introduced into the solution. The dihydrochloride salt, which precipitated on standing at +4° C., was filtered, dried and analysed. Analytical data for this material are as followed:

HPLC (Conditions: Column Bondapack C18 waters, 10 μm. 3.9×300 mm) showed a purity of 100% area as assessed by peak area.

Elemental analysis:

| | % C | % H | % N |
|---|---|---|---|
| Calculated: | 54.44 | 5.66 | 6.05 |
| Found: | 53.52 | 5.69 | 6.11 |

Mass spectrum: MH⁺=391 (dihydrochloride salt)

Melting point: 222-224° C.

DSC: Differential Scanning Calorimetry

| | |
|---|---|
| 20° C./min: | onset = 227.5° C. |
| 5° C./min: | onset = 215.7° C. |

Infra-Red: 2950 cm⁻¹ (nNH⁺); 1748 cm⁻¹ (vCO)

XRay powder diffraction: characteristic diffraction peaks are observed at 2θ values (±0.5): 7.000°; 8.095°; 12.000°; 13.645°; 14.085°; 14.315°; 14.870°; 16.460°; 17.295°; 18.255°; 18.755°; 19.470°; 20.575°; 20.890°; 21.660°; 22.210°; 22.890°; 23.390°; 24.210°; 24.580°; 25.130°; 26.775°; 27.855°; 28.815°; 29.820°; 30.255°; 31.460°; 32.145°; 32.890°; 33.830°; 34.695°; 35.940°; 38.135°; 39.670°; 43.065°; 44.335°; 46.210; 48.720.

1-23. (canceled)

24. A process for the manufacture of 2-(2-(4-(bis(4-fluorophenyl)methyl)-1-piperazinyl)ethoxy)acetic acids, amides and related derivatives of the general formula (I) 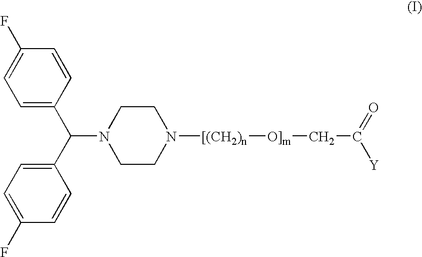
wherein:
Y represents hydroxy, or —NR¹R²; R¹ and R² each independently represent hydrogen or C₁₋₄ alkyl; m is 1 or 2, and n is 1 or 2, as well as non-toxic, pharmaceutically acceptable salts, and mixtures thereof which comprises
a) reacting compound of formula (II) 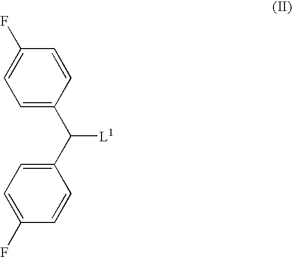
wherein L¹ represents a leaving group with a compound of formula (III) 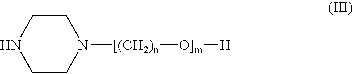
wherein n and m are defined as above, in the presence of a base and an inert solvent, and
b) reacting the corresponding compound of formula (IV) thus obtained 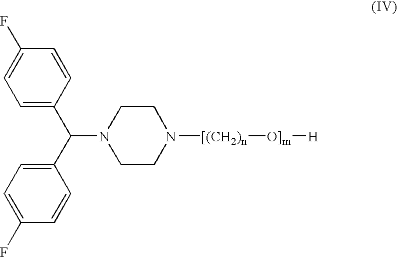
with a compound of formula (V) 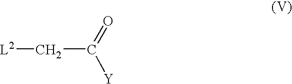
wherein L² represents a leaving group and Y is defined as above, in the presence of an inert solvent and a proton acceptor.

25. The process according to claim 24 wherein n is 2.

26. The process according to claim 24 wherein m is 1.

27. The process according to claim 24 wherein L¹ and L² represent, independently, halogen or a sulfonic ester group.

28. The process according to claim 24 wherein L¹ represents chlorine.

29. The process according to claim 24 wherein L² represents bromine.

30. The process according to claim 24 wherein the base in step (a) is selected from the group consisting of alkali metal carbonates, hydroxides and organic tertiary amines.

31. The process according to claim 30 wherein said base is sodium carbonate or potassium carbonate.

32. The process according to claim 24 wherein the proton acceptor in step (b) is selected from the group consisting of alkali metal hydrides, alkali metal hydroxides, alkali metal alkoxides and alkali metals.

33. The process according to claim 32 wherein said proton acceptor is sodium hydride or sodium methoxide.

34. The process according to claim 24 wherein the inert solvent is selected from the group consisting of aliphatic and aromatic hydrocarbons, ethers, amides and alcohols of low reactivity.

35. The process according to claim 34 wherein the inert solvent is hexane, toluene, methyl ethyl ketone (MEK), dimethoxyethane (DME), tetrahydrofuran (THF), dimethylformamide (DMF) or tert-butanol.

36. A process according to claim 24, wherein the compound obtained is a polymorphic form of the compound of formula (I) wherein n is 2, m is 1 and Y represents OH.

37. A compound obtained by the process according to claim 24.
